(19) United States
(12) Patent Application Publication
(10) Pub. No.: US 2012/0165580 A1
(43) Pub. Date: Jun. 28, 2012

(54) Process For Production of Polyolefins

(76) Inventors: Kenneth A. Bahler, Humble, TX (US); Aaron L. Wetterlind, League City, TX (US); Patrick R. Ford, Humble, TX (US); Derrick L. Laton, Houston, TX (US)

(21) Appl. No.: 13/351,338

(22) Filed: Jan. 17, 2012

Related U.S. Application Data
(63) Continuation-in-part of application No. 12/875,654, filed on Sep. 3, 2010.

(51) Int. Cl. C10M 105/00; C07C 2/00

(52) U.S. Cl. 585/10; 585/506

(57) ABSTRACT

A method for preparing a liquid polyolefin which includes contacting a feedstock comprising at least one olefin monomer with a catalyst system to produce a reactor effluent stream, filtering the reactor effluent stream, washing a created filter cake with a warm wash fluid comprising at least one hydrocarbon liquid, separating at least a portion of the wash fluid during the washing, sending said portion of the wash fluid through a distillation process, and recovering at least a portion of the liquid polyolefin that was in the filter cake. The catalyst system may be any conventional polyolefin catalyst system and, in any preferred embodiment of the invention, the catalyst system contains at least one activated metallocene catalyst. The reactor effluent stream comprises at least one liquid polyolefin, residual catalyst, and unreacted olefin monomer.

### PRIORITY CLAIM

This application is a continuation in part of U.S. application Ser. No. 12/875,654 filed on Sep. 3, 2010.

### FIELD OF THE INVENTION

The invention relates to an improvement in the process for the production of polyolefins, and specifically an improved filter cake wash system.

### BACKGROUND OF THE INVENTION

Efforts to improve upon the performance of mineral oil-based lubricants by the synthesis of oligomeric hydrocarbon fluids have been the subject of important research and development in the petroleum industry for at least fifty years. These efforts have led to the relatively recent market introduction of a number of synthetic lubricants. In terms of lubricant property improvement, the thrust of research efforts involving synthetic lubricants has been towards developing fluids exhibiting useful viscosities over a wide temperature range, i.e., improved viscosity index (VI), while also showing lubricities, thermal and oxidative stabilities and pour points equal to or better than those for mineral oils.

Poly alpha-olefins (PAOs) comprise a class of hydrocarbons manufactured by the catalytic oligomerization (polymerization to low-molecular-weight products) of linear alpha-olefin (LAO) monomers. These typically range from 1-octene to 1-dodecene, although oligomeric copolymers of lower olefins such as ethylene and propylene may also be used, including copolymers of ethylene with higher olefins as described in U.S. Pat. No. 4,956,122 and the patents referred to therein. PAO products have achieved importance in the lubricating oil market. Typically there are two classes of synthetic hydrocarbon fluids (SHF) produced from LAOs, the two classes being denoted as PAO and HVI-PAO (high viscosity index PAO). PAOs and HVI-PAOs of different viscosity grades may be produced using promoted BF₃ or AlCl₃ catalysts.

PAOs have found commercial success in the lubricant field for their superiority to mineral based lubricants. In terms of lubricant property improvement, research efforts have led to PAO fluids exhibiting useful viscosities over a wide range of temperatures, i.e., improved viscosity index, while also showing lubricity, thermal and oxidative stability and pour point equal to or better than mineral oil. These relatively new synthetic lubricants lower mechanical friction, enhancing mechanical efficiency over the full spectrum of mechanical loads and do so over a wider range of operating conditions than mineral oil lubricants.

The use of metallocene catalysts in oligomerization reactions is known, such as in WO 2007/011832, WO 2007/011459, and WO 2007/011973. PAOs made with metallocenes have yet to find wide applicability in the lubricant market, however, due to process inefficiencies and economics. This invention is intended to address such issues by providing an improved process for producing PAOs using metallocene catalysts. The advantages of this invention are not limited to metallocene-catalyzed oligomerization processes, however, and are useful in any conventional process to make polyolefins.

### SUMMARY OF THE INVENTION

This invention is directed toward an improved process to make a polyolefin product, and specifically an improved filter cake wash system. Disclosed herein is a method for preparing a liquid polyolefin, the method comprising: contacting a feedstock comprising at least one olefin monomer with a catalyst system to produce a reactor effluent stream; filtering the reactor effluent stream, washing a created filter cake with a warm wash fluid comprising at least one hydrocarbon liquid; separating at least a portion of the wash fluid off during and after the washing; sending said portion of the wash fluid through a distillation process; and recovering at least a portion of the liquid polyolefin that was trapped in the filter cake.

This process may further comprise the steps of, prior to filtering, contacting the reactor effluent stream with a deactivator to deactivate the residual catalyst to produce a deactivated reactor effluent stream, and contacting the deactivated reactor effluent stream with a sorbent, wherein the sorbent is capable of chemically and physically interacting with the residual catalyst. It may also comprise the steps of, after distilling the separated portion of the wash fluid, recycling the light olefins obtained from this wash fluid to the reactor for further conversion into product and/or recycling the heavy olefins obtained from this wash fluid to the filter for use as additional wash fluid.

Several process efficiencies and enhancements are achieved by this invention. First, recovery of polyolefin product from the filter cake is increased, thus improving overall product yield. Second, utilization of preferred feedstocks and wash fluids is improved by a unique wash fluid flash and distillation process, thus improving overall process efficiency and economics. Third, the process for disposing of the filter cake is made easier and more efficient by the use of a warm wash fluid stream instead of a hot stream. This keeps the filter cake cooler and thus easier and safer to handle. Additionally, this feature, along with the wash fluid distillation process, can maintain the flash point of the filter cake high enough such that the filter cake does not fall into an environmental waste classification that requires special handling and disposal measures. Lastly, this improved wash system can eliminate the need to use water to wash the filter, a method that creates a large amount of waste water that requires further processing.

### BRIEF DESCRIPTION OF THE FIGURES

This invention will be described with reference to the accompanying drawing wherein:

FIG. 1 is a simplified flow diagram of the manufacturing process in an embodiment of the invention.

### DETAILED DESCRIPTION OF THE INVENTION

This invention is directed toward an improved process to make a polyolefin, and specifically an improved filter cake wash system. While several embodiments of the invention will be specifically described, various other modifications will be apparent to and could be readily made by those skilled in the art in possession of this disclosure without departing from the spirit and scope of the invention. Accordingly, it is not intended that the claims be limited to the examples or descriptions set forth but rather that the claims be construed as encompassing all features of patentable novelty in the present invention, including all features which would be treated as equivalents by those skilled in the art.

### Feedstocks

Polyolefins are manufactured by the catalytic oligomerization of olefins, preferably alpha-olefins, and preferably LAO monomers. Useful alpha-olefins are typically selected from C₅ to C₃₀ alpha-olefins and any mixture thereof. By “mixture” of alpha-olefins, it is meant that at least two different alpha-olefins are present in the feed. In embodiments of the invention where the feed is a mixture, the feed may comprise anywhere from 2 to 25 different alpha-olefins. Thus, the feed may comprise at least two, or at least three, or at least four, or at least five, or at least six, or at least seven, or at least eight, and so on, different feeds. The embodiments of the invention may be further characterized by having no single alpha-olefin present in an amount greater than 80 wt %, or 60 wt %, or 50 wt %, or 49 wt %, or 40 wt %, or 33 wt %, or 30 wt %, or 25 wt %, or 20 wt %.

The amounts of a particular alpha-olefin present in a feed will be specified herein as percent by weight of the entire amount of alpha-olefin in the feed, unless otherwise specified. Thus, it will be recognized that the feed may also comprise an inert (with respect to the oligomerization reaction in question) material, such as a carrier, a solvent, or other olefin component that is not an alpha-olefin. Examples are propane, n-butane, iso-butane, cis- or trans-2-butenes, iso-butenes, and the like, that may be present with propylene or 1-butene feed. Other examples are the impurity internal olefins or vinylidene olefins that are present in the alpha-olefin feed.

Feeds may be advantageously selected from C₅ to C₃₀, C₅ to C₂₄, C₅ to C₁₈, C₅ to C₁₆, C₅ to C₁₄, C₆ to C₂₀, C₆ to C₁₈, C₆ to C₁₆, or C₆ to C₁₄ alpha-olefins, among other possible feed sources, such as any lower limit listed herein to any upper limit listed herein. In other embodiments of the invention, the feed will comprise at least one monomer selected from propylene, 1-butene, 1-pentene, 1-hexene to 1-heptene and at least one monomer selected from C₁₂-C₁₈ alpha-olefins. In any embodiment of the invention, the amount of ethylene in the feed is not more than 10 mol %.

When employing a mixed feed, one acceptable mixed feed is any mixture of 1-hexene, 1-octene, 1-decene, 1-dodecene, and 1-tetradecene. Mixtures in all proportions may be used, e.g., from about 1 wt % to about 90 wt % 1-hexene, from about 1 wt % to about 90 wt % 1-octene, from about 1 wt % to about 90 wt % 1-decene, from about 1 wt % to about 90 wt % 1-dodecene, and from about 1 wt % to about 90 wt % tetradecene. In preferred embodiments of the invention, 1-hexene is present in the amount of about 1 wt %, 2 wt %, 3 wt %, 4 wt % or 5 wt % to about 10 wt % or 20 wt %, 1-octene is present in the amount of 40 wt %, 50 wt %, or 60 wt % to about 70 wt %, 75 wt %, 80 wt %, 85 wt %, 90 wt %, or 95 wt %, 1-decene is present in the amount of about 25 wt %, 30 wt %, 40 wt % or 50 wt % to about 60 wt %, 70 wt % or 75 wt %, 1-dodecene is present in the amount of about 10 wt %, 20 wt %, 25 wt %, 30 wt % or 40 wt % to about 45 wt %, 50 wt % or 60 wt %, and 1-tetradecene is present in the amount of 1 wt %, 2 wt %, 3 wt %, 4 wt %, 5 wt %, 10 wt %, 15 wt %, 20 wt % or 25 wt % to about 30 wt %, 40 wt %, or 50 wt %. Ranges from any lower limit to any higher limit just disclosed are contemplated, e.g., from about 3 wt % to about 10 wt % 1-hexene or from about 2 wt % to about 20 wt % 1-hexene, from about 40 to 95% 1-octene, from about 25 wt % to about 70 wt % 1-decene or from about 40 wt % to about 70 wt % 1-decene, from about 10 wt % to about 45 wt % 1-dodecene or from about 25 wt % to about 50 wt % 1-dodecene, and from about 5 wt % to about 30 wt % 1-tetradecene or from about 15 wt % to about 50 wt % 1-tetradecene. Numerous other ranges are contemplated, such as ranges plus or minus 5% (±5%) from those specified in the examples.

In an embodiment of the invention, the mixed feed (or mixture of alpha-olefins contacting the oligomerization catalyst and promoters) consists essentially of 1-hexene, 1-octene, 1-decene, 1-dodecene, and/or 1-tetradecene, wherein the phrase “consists essentially of” (or “consisting essentially of” and the like) takes its ordinary meaning, so that no other alpha-olefin is present (or for that matter nothing else is present) that would affect the basic and novel features of the present invention. In yet another preferred embodiment of the invention, the mixed feed consists of 1-hexene, 1-octene, 1-decene, 1-dodecene, and/or 1-tetradecene, meaning that no other olefin is present (allowing for inevitable impurities).

Another mixed feedstock useful in the present invention is a mixed feed of 1-hexene, 1-decene, and 1-tetradecene. Mixtures in all proportions may be used, e.g., from about 1 wt % to about 90 wt % 1-hexene, from about 1 wt % to about 90 wt % 1-decene, and from about 1 wt % to about 90 wt % 1-tetradecene. In preferred embodiments, the 1-hexene is present in amounts of 1 wt %, 2 wt %, 3 wt %, 4 wt % or 5 wt % to about 10 wt %, 20 wt %, 25 wt % or 30 wt %, 1-decene is present in the amount of about 25 wt %, 30 wt %, 40 wt % or 50 wt % to about 60 wt %, 70 wt % or 75 wt %, and 1-tetradecene is present in the amount of 1 wt %, 2 wt %, 3 wt %, 4 wt %, 5 wt %, 10 wt %, 15 wt %, 20 wt % or 25 wt % to about 30 wt % or 40 wt %. Ranges from any lower limit to any higher limit just disclosed are contemplated.

Mixed feedstocks of two LAOs are also contemplated by the present invention. Such two component feedstocks may be blends of 1-hexene and 1-decene, 1-hexene and 1-dodecene, 1-decene and 1-dodecene, 1-decene and 1-tetradecene, or 1-dodecene and 1-tetradecene. For such two LAO mixed feedstocks, either component may be present in amounts of 1 wt % to 99 wt %, with preferred ranges for both components being in the ranges of 10 wt % to 90 wt %, 15 wt % to 85 wt %, 20 wt % to 80 wt %, or 30 wt % to 70 wt %.

In other embodiments of the invention the olefin feed consists essentially of a single LAO such as 1-decene, 1-dodecene, or the like.

Particularly advantageous feedstocks include alpha-olefins derived from an ethylene growth process, from Fischer-Tropsch synthesis, from steam or thermal cracking processes, syn-gas synthesis, C₄ stream containing 1-butene from refinery operation, such as Raff-1 or Raff-2 stream, and so forth. The alpha-olefin made from ethylene growth processes contains only even-number olefins. Alpha-olefins containing both even- and odd-number olefins can also be made from steam cracking or thermal cracking of wax, such as petroleum wax, Fischer-Tropsch wax, or any other readily available hydrocarbon wax. Alpha-olefins can also be made in a Fischer-Tropsch synthesis process. Alpha-olefins can also be made directly from syngas synthesis processes, which can produce significant amounts of C₃-C₁₅ alpha-olefins, containing both even- and odd-number olefins.

In an embodiment of the invention, it is advantageous to use a high quality feed with minimal inert material. However, alpha-olefins containing other inert components, including saturated hydrocarbons, internal or vinylidene olefins or aromatic diluents can also be used as feed. In this case, the alpha-olefin would be reacted to give polymer and inert components that will be passed through the reactor unaffected. The polymerization process is also a separation process.

In an embodiment of the invention, the olefins used in the feed are co-fed into the reactor. In an embodiment of the invention, the olefins are fed separately into the reactor. In either case, the catalyst/promoters may also be feed separately or together, with respect to each other and with respect to the alpha-olefin species.

### Catalyst System

The catalyst system may be any catalyst system capable of producing polyolefins. The term “catalyst system” is defined herein to mean a catalyst precursor/activator pair. When “catalyst system” is used to describe such a pair before activation, it means the unactivated catalyst (precatalyst) together with an activator and, optionally, a co-activator (such as a trialkyl aluminum compound). When it is used to describe such a pair after activation, it means the activated catalyst and the activator or other charge-balancing moiety. Furthermore, this activated “catalyst system” may optionally comprise the co-activator and/or other charge-balancing moiety.

In any preferred embodiment of the invention, the catalyst system contains at least one activated metallocene catalyst. In such embodiments of the invention, the catalyst system comprises a metallocene compound (selected from one or more compounds according to Formula 1, below) together with an activator, optionally a co-activator, and optionally a scavenger.

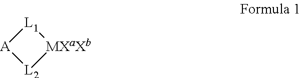

### In Formula 1:

M is selected from Group 4 transition metals, preferably zirconium (Zr), hafnium (Hf), and titanium (Ti),

L₁ and L₂ are independently selected from cyclopentadienyl (“Cp”), indenyl, and fluorenyl, which may be substituted or unsubstituted, and which may be partially hydrogenated,

A is an optional bridging group which if present, is preferably selected from dialkylsilyl, dialkylmethyl, ethenyl (—CH₂—CH₂—), alkylethenyl (—CR₂—CR₂—), where alkyl can be independently hydrogen radical, C₁ to C₁₆ alkyl radical or phenyl, tolyl, xylyl radical and the like, and

X^{a} and X^{b}, are independently selected from halides, OR(R is an alkyl group, preferably selected from C₁ to C₅ straight or branched chain alkyl groups), hydrogen, C₁ to C₁₆ alkyl or aryl groups, haloalkyl, and the like.

In using the terms “substituted or unsubstituted cyclopentadienyl ligand,” “substituted or unsubstituted indenyl ligand,” and “substituted or unsubstituted tetrahydroindenyl ligand,” “substituted or unsubstituted fluorenyl ligand,” and “substituted or unsubstituted tetrahydrofluorenyl or octahydrofluorenyl ligand,” the substitution to the ligand may be hydrocarbyl, substituted hydrocarbyl, halocarbyl, substituted halocarbyl, silylcarbyl, or germylcarbyl. The substitution may also be within the ring giving heterocyclopentadienyl ligands, heteroindenyl ligands or heterotetrahydroindenyl ligands, each of which can additionally be substituted or unsubstituted.

The terms “hydrocarbyl radical,” “hydrocarbyl,” and “hydrocarbyl group” are used interchangeably throughout this document. Likewise the terms “group,” “radical,” and “substituent” are also used interchangeably. For purposes of this invention, a “hydrocarbyl radical” is a C₁-C₁₀₀ radical that may be linear, branched, or cyclic, and when cyclic, aromatic or non-aromatic, and include substituted hydrocarbyl radicals, halocarbyl radicals, and substituted halocarbyl radicals, silylcarbyl radicals, and germylcarbyl radicals as these terms are defined below. Substituted hydrocarbyl radicals are radicals in which at least one hydrogen atom has been substituted with at least one functional group.

Halocarbyl radicals are radicals in which one or more hydrocarbyl hydrogen atoms have been substituted with at least one halogen (e.g., F, Cl, Br, I) or halogen-containing group (e.g., CF₃). Substituted halocarbyl radicals are radicals in which at least one halocarbyl hydrogen or halogen atom has been substituted with at least one functional group.

Silylcarbyl radicals (also called silylcarbyls) are groups in which the silyl functionality is bonded directly to the indicated atom or atoms. Germylcarbyl radicals (also called germylcarbyls) are groups in which the germyl functionality is bonded directly to the indicated atom or atoms. Polar radicals or polar groups are groups in which the heteroatom functionality is bonded directly to the indicated atom or atoms. They include heteroatoms of groups 1-17 of the Periodic Table either alone or connected to other elements by covalent or other interactions such as ionic, van der Waals forces, or hydrogen bonding.

Catalysts such as 1,2,3,4-tetramethylcyclopentadienylzirconium dichloride, 1,2,4-trimethylcyclopentadienylzirconium dichloride, or pentamethylcyclopentadienyl zirconium dichloride or their dialkyl analogs are preferred. Certain bridged and bridged with substitution catalysts, such as the di-halides or dialkyls of dimethylsilylbis[indenyl]zirconium or dimethylsilylbis[tetrahydro-indenyl]zirconium dimethylsilylbis[1-methylindenyl]zirconium or their hafnium analogs, etc. are also desirable.

### Activators/Co-Activators

Activators that may be used include aluminoxanes such as methyl aluminoxane, modified methyl aluminoxane, ethyl aluminoxane, iso-butyl aluminoxane and the like, or non-coordinating anions (NCAs) such as Lewis acid activators including triphenyl boron, tris-perfluorophenyl boron, tris-perfluorophenyl aluminum and the like, or ionic activators including dimethylanilinium tetrakis perfluorophenyl borate, triphenyl carbonium tetrakis perfluorophenyl borate, dimethylanilinium tetrakis perfluorophenyl aluminate, and the like.

For purposes of this invention and the claims thereto, a non-coordinating anion (NCA) is an anion which either does not coordinate to the catalyst metal cation or that coordinates only weakly to the metal cation. An NCA coordinates weakly enough that a neutral Lewis base, such as an olefinically or acetylenically unsaturated monomer, can displace it from the catalyst center. Any metal or metalloid that can form a compatible, weakly coordinating complex with the catalyst metal cation may be used or contained in the NCA. Suitable metals include, but are not limited to, aluminum, gold, and platinum. Suitable metalloids include, but are not limited to, boron, aluminum, phosphorus, and silicon. A subclass of NCAs comprises stoichiometric activators, which can be either neutral or ionic. The terms ionic activator, and stoichiometric ionic activator can be used interchangeably. Likewise, the terms neutral stoichiometric activator and Lewis acid activator can be used interchangeably.

A co-activator is a compound capable of alkylating the transition metal complex, such that when used in combination with an activator, an active catalyst is formed. Co-activators include aluminoxanes such as methyl aluminoxane, modified aluminoxanes such as modified methyl aluminoxane, and trialkyl aluminums such as trimethyl aluminum, tri-isobutyl aluminum, triethyl aluminum, and tri-isopropyl aluminum, tri-n-hexyl aluminum, tri-n-octyl aluminum, tri-n-decyl aluminum or tri-n-dodecyl aluminum. Co-activators are typically used in combination with Lewis acid activators and ionic activators when the pre-catalyst is not a dihydrocarbyl or dihydride complex. Sometimes co-activators are also used as scavengers to deactivate impurities in feed or reactors.

When the catalyst system is metallocene plus methylalumoxane, the range of methylalumoxane used is typically in the range of 0.1 milligram (mg) to 500 mg/g of alpha-olefin feed. A more preferred range is from 0.05 mg to 10 mg/g of alpha-olefin feed. Furthermore, the molar ratios of the aluminum to metallocene (Al/M molar ration) range are from 2 to 4000, preferably 10 to 2000, more preferably 50 to 1000, preferably 100 to 500. When the catalyst system is metallocene plus a Lewis Acid or an ionic promoter with NCA component, the metallocene use is typically in the range of 0.01 microgram to 500 micrograms of metallocene component per gram of alpha-olefin feed, preferably 0.1 microgram to 100 microgram of metallocene component per gram of alpha-olefin feed. Furthermore, the molar ratio of the NCA activator to metallocene is in the range from 0.1 to 10, preferably 0.5 to 5, preferably 0.5 to 3. If a co-activator of alkylaluminum compound is used, the molar ratio of the Al to metallocene is in the range from 1 to 1000, preferably 2 to 500, preferably 4 to 400.

Other components used in the reactor system can include inert solvent, catalyst diluent, etc. These components can also be recycled during the operation.

### Polymerization Process

While polymerization reaction conditions may generally be determined by one of ordinary skill in the art in possession of this disclosure, typical conditions are discussed below. While it is recognized that oligomerization generally refers to the conversion of monomers to a finite degree of polymerization, the terms “polymerization” and “oligomerization” are used interchangeably in this disclosure and the advantages of this invention are useful in either type of reaction.

The polymerization process will typically occur in a homogeneous or colloidal solution process. In an embodiment of the invention, this involves polymerization in a continuous reactor in which the starting feed, catalyst materials, and polymer formed are agitated to reduce or avoid concentration or temperature gradients. In other embodiments of the invention, the process can be advantageously carried out in a conventional continuous stirred tank reactor (CSTR), a batch reactor, or plug flow reactor, or more than one reactor operated in series or parallel. These processes may also be employed in a single reactor, as well as parallel or series reactor configurations. A typical CSTR has an internal agitator at the bottom of the reactor to continuously mix the reactor contents. However, constant movement of the reactor contents in a tank reactor may be achieved by other means that do not require an internal agitator, such as spray nozzles or a combination of spray nozzles and internal baffles.

If desired, temperature may be controlled in any reactor by balancing the heat of polymerization with reactor cooling via reactor jackets or cooling coils, a cooled side-stream of reactant to cool the contents of the reactor, auto refrigeration, pre-chilled feeds, vaporization of liquid medium (diluent, monomers, or solvent), or any combination of these methods. Adiabatic reactors with pre-chilled feeds may also be used. The optimal reactor temperature depends on the catalyst used and the product desired. Higher temperatures tend to give lower molecular weights and lower temperatures tend to give higher molecular weights, however this is not an absolute rule. In general, the reactor temperature can vary between about 0° C. and about 300° C., preferably from about 10° C. to about 250° C., and most preferably from about 25° C. to about 230° C. Usually, it is important to control the reaction temperature. It is useful to have minimum temperature fluctuation over the course of the reaction to produce fluids with narrow molecular weight distribution (MWD), which will result in products with higher shear stability. If multiple reactors are used in series or in parallel, it is also useful to keep the temperature constant if narrow MWD is desired. If it is desired to produce fluids with broad MWD, one can adjust the reaction temperature fluctuation, or in series operation, the second reactor temperature can be higher than the first reactor temperature. In parallel reactor operation, the temperatures of the two reactors are independent. One can also affect MWD of the products by using two different types of metallocene catalysts.

The pressure in any reactor can vary typically from about 0.1 atmosphere to 100 atmosphere (10 kPa to 10,100 kPa), preferably from 0.5 atm to 75 atm (50 kPa to 7600 kPa), and most preferably from 1.0 atm to 50 atm (101 kPa to 5066 kPa). The reaction can be carried out under the atmosphere of nitrogen or hydrogen. A small amount of hydrogen may be added to the reactor to improve the catalyst productivity. The amount of hydrogen is preferred to be kept at a high enough level to improve catalyst productivity, but not high enough to introduce hydrogenation of olefins, especially the feed olefins because the conversion of alpha-olefins into saturated paraffins is very detrimental to the efficiency of the process. The amount of hydrogen partial pressure is preferably less than 200 psi (1379 kPa), preferably less than 150 psi (1034 kPa), preferably less than 100 psi (689 kPa), preferably less than 50 psi (345 kPa), preferably less than 25 psi (172 kPa), preferably less than 10 psi (69 kPa), preferably less than 5 psi (34 kPa), and preferably less than 1 psi (6.9 kPa). In a particularly preferred embodiment in any of the processes described herein the concentration of hydrogen in the reactant phase is less than 200 ppm, preferably less than 100 ppm, preferably less than 50 ppm, preferably less than 10 ppm, and preferably less than 1 ppm.

The reaction time or reactor residence time is usually dependent on the catalyst used, amount of catalyst system used, and desired conversion level. Usually the amount of catalyst system used is determinative. Higher catalyst loading tends to gives higher conversion in a shorter reaction time. However, at a point, high catalyst loading also makes it difficult to manage reaction heat or temperature and makes the process uneconomical. Therefore, it is useful to choose a catalyst with maximum catalyst productivity to minimize the amount of catalyst system needed. Desirable residence times may be determined by one of ordinary skill in the art in possession of this disclosure, and will typically range from 1 minute to 20 hours, or more typically 5 minutes to 10 hours. See, for instance, U.S. Pat. No. 5,705,577 for typical process conditions.

Typically it is preferred to have the highest possible conversion of feed alpha-olefin in the shortest possible reaction time. However, in CSTR operation, sometimes it is optimal to run the reaction at slightly less than 100% conversion. There are also occasions when partial conversion is desirable, typically when the narrowest possible MWD of the product is desired, because partial conversion can avoid a MWD broadening effect. If the reaction is conducted to less than 100% conversion, the unreacted feedstock materials, after separation from materials, can be recycled to increase the total process efficiency.

The reaction process comprises contacting olefin monomers with the catalyst system, preferably in a suitable diluent, solvent, recycle, or mixture thereof, and allowing the reaction to occur for a sufficient time to produce the desired polymers or oligomers. Suitable diluents or solvents include both aliphatic and aromatic hydrocarbons. Aromatics such as benzene, toluene, xylenes, ethylbenzene, propylbenzene, cumene, t-butylbenzene are suitable. Alkanes, such as hexane, heptane, pentane, isopentane, and octane, Norpar™ fluids or Isopar™ fluids from ExxonMobil Chemical Company in Houston, Tex. are also suitable. Toluene is a preferred substance for dissolving catalyst components. Norpar™ fluids, Isopar™ fluids, or hexanes (or mixtures thereof) are preferred as reaction diluents. Oftentimes, a mixture of toluene and Norpar™ or Isopar™ fluids is used as a diluent or solvent.

### Polyolefin Isolation

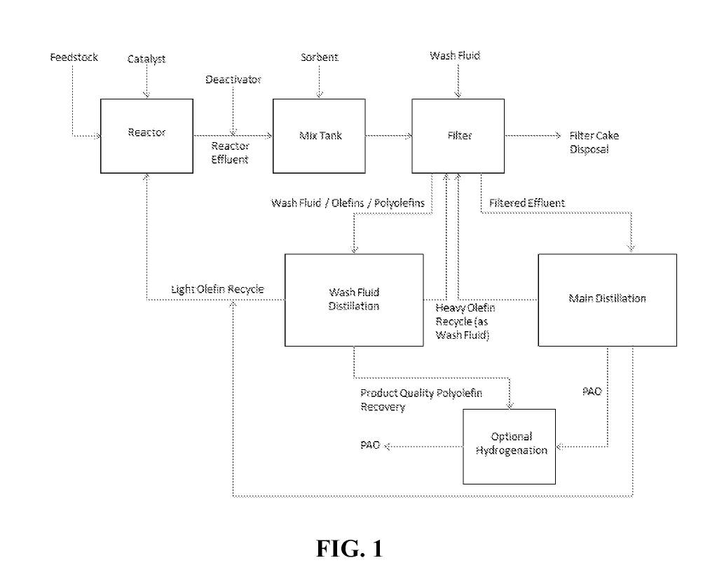

When the polymerization reaction is progressed to the desired conversion, the reactor effluent is withdrawn from the reactor, see FIG. 1. The reactor effluent comprises at least one liquid polyolefin, residual catalyst, and unreacted olefin monomer, and may further comprise compounds considered inerts such as internal olefins, branched olefins, and paraffins that entered the system via the feedstock or reaction in the reactor. The term “residual catalyst” as used herein will include unreacted catalyst precursor, if any, unreacted activated catalyst and various forms of the catalyst which may be formed during the reaction, as well as any unused co-activator. The reactor effluent may also contain one or more other diluents or solvents, and scavengers added to the reactor.

Prior to or concurrent with entry into a mix tank, the reactor effluent may be treated to deactivate the residual catalyst and, if necessary, any co-activator and scavenger. Deactivation is typically accomplished by introduction of air, CO₂, water or other deactivator. This may be either in an adjacent deactivation vessel or the deactivation agent may be fed into the effluent pipe under conditions of static mixing. The reactor effluent comprising the deactivated residual catalyst may be referred to as “deactivated effluent.”

In an embodiment of the invention, removal of the residual catalyst from the deactivated effluent begins in the mix tank, see FIG. 1. At least one sorbent having an active surface area and pore volume capable of adsorbing the catalyst components is mixed with the deactivated effluent. Typical examples of suitable solid sorbents are natural or synthetic clay, modified clay, diatomaceous earth, activated charcoal, silica gel, alumina, aluminosilicate, zeolites, molecular sieves, cellulose material, metal oxides or metal salts, such as calcium oxides, magnesium oxides, titanium oxides, zirconium oxides, aluminum oxides, activated or treated in appropriate manners. Generally the sorbent should have a surface area greater than 0.1 m²/gram and a pore volume of greater than 0.01 cc/gram. The sorbent may have both chemical and physical active sites to interact with the catalyst components. Such solid sorbents having surface hydroxyl or oxygen groups thereon yield chemical reactions with the catalyst components thereby providing strong sorption of the catalyst components and facilitating the high degree of catalyst removal. The sorbent may also act as catalyst poison to deactivate the polymerization reaction. If the sorbent contains a sufficient amount of water or oxygen on its molecular surface, no extra catalyst deactivator may be needed prior to treating the reactor effluent with the solid sorbent.

The sorbent may be introduced to the deactivated effluent in either dry form or it may be pre-blended with a hydrocarbon fluid to form a sorbent slurry. When slurried, the hydrocarbon fluid may be any suitable non-reactive hydrocarbon fluid. A “non-reactive hydrocarbon fluid” as used herein is a fluid that does not react with the sorbent or any components of the deactivated effluent stream. The hydrocarbon fluid may be any C₅ to C₃₀ fluid, and is preferably selected to correspond to one of the fluids that was used either as a feedstock to the reactor or a catalyst diluent.

Separation of the solid sorbent with the catalyst adsorbed thereon from the remaining deactivated effluent is easily accomplished. After the sorbent is mixed with the deactivated effluent, the mixture is passed through a filter system, see FIG. 1, such as a membrane filter or any suitable commercial filter, which may be packed with filter aide or other solid material which functions both as filter aide and an additional catalyst sorbent. Examples of suitable filtering systems include a filter drum or a solid filter bed.

One example of a useful filter system is a pressure leaf filter, such as a FUNDA® pressure leaf filter drum. The individual filter leafs are precoated with a sorbent prior to introduction of the deactivated effluent. Precoating is done with a sorbent slurry which settles on the filter leafs as the slurry vertically flows through the drum. The slurry is a blend of a solid sorbent and a hydrocarbon fluid. The solid sorbent may or may not be identical to the solid sorbent used in the mix tank in the first step of catalyst removal. Particularly preferred sorbents include silica, alumina, activated alumina, di-atomaceous earth filter aid, zeolites of different pore size, MCM41, natural or synthetic clay materials, micro crystalline material or powdered cellulose material with hydroxyl group. The sorbent can be used by itself or mixed with other sorbents. The hydrocarbon fluid in the sorbent slurry may be any inert C₅ to C₃₀ fluid, and is preferably selected to correspond to one of the fluids that was used either as a feedstock to the reactor or a catalyst diluent. In one embodiment of the invention, the hydrocarbon fluid used in the sorbent slurry is the same as used in the mix tank and is also unreactive with the sorbent and components of the deactivated effluent stream.

Filtering may be either a continuous or batch operation. The factors to consider in determining optimal mode include a) type of filter; b) filter capacity; c) reactor capacity; d) reactor operation mode; and e) mix tank capacity, and such determination can be made by one of ordinary skill in the art in possession of this disclosure. After filtering, the filtered liquid flows from a discharge valve at the bottom of the filter drum. The filtered liquid contains polyolefin product, unreacted monomers, hydrocarbon liquids used as diluents, catalyst deactivator, and/or sorbent slurry liquid. The filtered liquid may also contain inerts introduced via the feedstream or unintentionally generated in the reactor.

As the deactivated effluent passes through the filter, catalyst is retained in the filter. After a period, catalyst solids build up on the filter components. These solids, known as the “filter cake,” must periodically be removed to effectively continue filtration. Typically, the filter cake is removed from the filter and the sorbent in the filter cake could then be reactivated and regenerated by calcinations or by washing/drying/calcinations. The reactivated solid sorbent could then be re-used in the system. The washing of the filter cake in this step may be accomplished using water, however, this creates a large amount of waste water, which requires additional processing. This use of water to wash the filter, and thus the creation of a significant amount of waste water, is eliminated in favor of an improved wash process, as explained below.

Although the filter cake, even after draining the filtered liquid, may appear dry, due to the highly sorbent nature of the solids, the cake may actually contain up to 75% liquid. This liquid will have a fractional content about equivalent to the liquid exiting the reactor. Thus, if the reactor effluent is 90 vol % polyolefin product and 10 vol % other materials, the liquid trapped in the filter cake will also be about 90 vol % polyolefin product. Thus, prior to removal of the filter cake from the filter, the filter cake is washed with a wash fluid to recover this product. The wash fluid forces the liquid out of the filter cake, in effect replacing this liquid comprising polyolefin product in the filter cake with the wash fluid.

In known processes, when a closed loop wash stream is used to wash the filter cake, the stream has to be heated to separate the unreacted olefins from the polyolefin product. When such a heated stream is cycled through the filter, the washed filter cake becomes so hot that special personal protective equipment is required for personnel handling the filter cake. Additionally, especially if the olefin monomers used in the feedstocks and wash fluids are lighter olefins (which are often preferred feedstocks for making PAOs), the washed filter cake absorbs a sufficient amount of these materials to lower its flash point such that it may require additional handling and disposal measures due to its environmental waste classification.

This invention addresses these issues in a unique way. First, a warm wash stream, instead of a hot stream, is used. For purposes of this disclosure, “warm” is defined as a temperature of about 25° C. to 60° C., although temperatures within plus or minus 5° C. of this range are contemplated. The warm wash stream provides an advantage over known processes in that the filter cake remains at a lower temperature, providing for safer, easier, and more efficient disposal. Second, a wash fluid distillation process (see FIG. 1) is added. This wash fluid distillation may comprise a distillation tower, flash drum, nitrogen sparging, and/or any other suitable equipment and process steps known to one skilled in the art to be useful in distillation processes. During the filter wash, a portion of the used wash fluid is taken off and sent through this wash fluid distillation which comprises heating the wash fluid to an appropriate temperature to cause at least a portion of the fluid to flash overhead. This distillation separates the lower flash point olefins from the heavier olefins and polyolefin product. At least a portion of the lower flash point olefins may be recycled back into the reactor for further conversion into product. At least a portion of the heavier olefins and polyolefin product stream may be sent to the main distillation facility to separate the product quality polyolefins from the remaining olefins. The remaining olefins from the main distillation may then be cooled and sent back to the filter drum as wash fluid.

This wash fluid distillation improves and optimizes the process in several ways. First, it optimizes the recovery of polyolefin product from the filter cake and the utilization of preferred feedstocks and wash fluids, as not only is polyolefin product recovered from the wash fluid, but both the light and heavy olefins recovered from the wash fluid may be recycled to the reactor and/or filter. Second, in an embodiment of the invention, the wash fluid starts out as 100% pure or nearly 100% pure olefin monomer, and is a monomer with a flash point above the threshold of what requires any special environmental waste classification. As the wash fluid is used and building up in the filter cake, the fluid is continuously absorbing olefins from the effluent, which may include low flash point unreacted olefins from the feedstocks. The wash fluid distillation, where wash fluid is continuously taken off and lower flash point olefins are removed during the course of the wash, allows the concentration of the wash fluid, including the concentration of lower flash point olefins, to reach a steady state. This steady state concentration can be set such that it stays below a threshold that will require the filter cake on disposal to have any special environmental waste classification. This steady state concentration depends on the particular wash fluids used and other factors, and can be set and controlled by one of ordinary skill in the art in possession of this disclosure.

The wash fluid prior to use is at least one hydrocarbon liquid and may be any C₅ to C₃₀ fluid; and is preferably selected to correspond to a fluid or monomer that forms part of the feedstock to the reactor, and preferably feedstocks with higher flash points. In an embodiment of the invention, the wash fluid prior to use is 100% C₁₄, 100% C₁₂, or 100% C₁₀. In an embodiment of the invention, the wash fluid prior to use comprises 90 vol % to 100 vol % C₁₄, 90 vol % to 100 vol % C₁₂, 90 vol % to 100 vol % C₁₀, or 90 vol % to 100 vol % of any mixture of these monomers. By employing only feedstock liquids as the wash fluid, the used wash fluid may be recycled to the reactor and thus the overall consumption of hydrocarbon liquids and other liquids in the system is reduced. In an embodiment of the invention, the wash fluid prior to use comprises 90 vol % to 100 vol % of any C₅ to C₃₀ hydrocarbon fluid and may also contain a minor amount of other fluids such as C₂-C₅ hydrocarbons, inert liquids such as those in any of the reactors, byproducts formed in the reaction, or water. In an embodiment of the invention, the wash fluid prior to use contains no more than 5.0 vol % of water, no more than 1.0 vol % of water, or no more than 0.1 vol % of water.

In an embodiment of the invention, the steady state concentration of the wash fluid is at least 50 wt %, at least 55 wt %, at least 60 wt %, or at least 65 wt % of at least one hydrocarbon liquid selected from the group comprising C₁₄, C₁₂, or C₁₀. In an embodiment of the invention, the steady state concentration of the wash fluid is less than 20 wt %, preferably less than 15 wt %, preferably less than 10 wt %, preferably less than 7.5 wt %, and preferably less than 5 wt % polyolefin product. In an embodiment of the invention, the steady state concentration of the wash fluid is less than 10 wt %, preferably less than 5 wt %, preferably less than 2.5 wt %, preferably less than 1 wt %, and preferably less than 0.5 wt % of C₂-C₆ hydrocarbons.

Wash fluids in addition to those disclosed above are contemplated as suitable for this invention. In an embodiment of the invention, the wash fluid may comprise water or steam in whole or in part, but additional equipment would be required, for example, equipment to treat the used water and an oil/water separator. Thus while water is suitable, it is an advantage of this invention to not use or require it because additional equipment and the associated expense is avoided. In an embodiment of the invention, nitrogen or a heavier hydrocarbon stream than what is disclosed above may also be used as wash fluids, although these options may also require additional equipment and/or process steps recognizable to one of ordinary skill in the art, such as additional heating and cooling facilities, compression facilities, etc.

The remaining filter cake solids comprise a blend of inert catalyst material and sorbent and residual hydrocarbon liquid. The remaining solids may be disposed of or the sorbent may be reactivated to remove the inert catalyst and returned to the system for further separation of catalyst from the reactor effluent. Prior to disposal of the used filter cake, it is preferable to remove as much of the valuable polyolefin and used wash fluid as possible. In an embodiment of the invention, the amount of polyolefin product remaining in the filter cake after washing is less than 20 wt %, preferably less than 15 wt %, preferably less than 10 wt %, preferably less than 7.5 wt %, or preferably no more than 5 wt %.

### Product Produced

Depending on the reaction conditions, the polymer product may have a high degree of unsaturation according to the bromine number as measured by ASTM D1159, or an equivalent method. The heavy oligomer fraction may be subjected to a hydrofinishing step to reduce the bromine number, usually to less than 3, less than 2, or less than 1, depending on hydrofinishing conditions and the desired application. Details on typical hydrogenation processes can be found in many published patents and literatures. Sometimes, when the polyolefin products have very high molecular weight or hydrogen is used during the polymerization step, the isolated polyolefin products will naturally have very low bromine number or degree of unsaturation and the product can be used directly in many applications without a separate hydrogenation step.

The light fraction, as separated directly from the reactor effluent or further fractionated from the initially separated light fraction, contains unreacted olefin monomers. This light fraction can be recycled, with or without any purge, into the polymerization reactor for further conversion into lube product. This fraction as is, or the appropriated fractions, can also be recycled into the polymerization reactor after passing through a feed pre-treatment column containing the typical polar component removing agents, such as activated alumina, molecular sieve, or other active sorbents. This pre-treatment column can remove any impurity from the catalyst residue or other impurities. Alternatively, this fraction can be combined with fresh feed olefins before the feed purification column.

The oligomerization product comprises a liquid polyolefin. The types of liquid polyolefins produced may include ethylene-alpha-olefin copolymer or terpolymer, homopolymer/copolymer/terpolymer of other alpha-olefins, linear alpha olefin (LAO) homopolymer/copolymer/terpolymer, etc. Specific examples of these polymers include PAOs, poly-1-decene, copolymer or terpolymer or multi-component liquid polymer of C₃ to C₂₄, terpolymer of C₈, C₁₀, C₁₂-LAO, copolymer of C₆ and C₁₂ or C₆ and C₁₄, copolymer of C₄ and C₁₂ or C₄ and C₁₄, ethylene-propylene copolymer, ethylene-butene copolymer, ethylene-pentene copolymer, ethylene-propylene-butene terpolymer, ethylene-propylene-hexene terpolymer, etc.

In an embodiment of the invention, any of the polyolefins produced have an M_{W} (weight average molecular weight) of 100,000 or less, preferably between 200 and 80,000, more preferably between 250 and 60,000, more preferably between 280 and 50,000, and most preferably between 336 and 40,000 g/mol. (Preferred M_{w}'s include those from 224 to 55,100, preferably from 392 to 30,000, more preferably 800 to 24,000, and most preferably 2,000 to 37,500 g/mol. Alternately preferred M_{w}'s include 224 to about 6790, and preferably 224 to about 2720).

In an embodiment of the invention, any of the polyolefins produced by the method herein have a number average molecular weight (Mₙ) of 50,000 or less, more preferably between 200 and 40,000, more preferably between 250 and 30,000, or most preferably between 500 and 20,000 g/mol. More preferred Mₙ ranges include 280 to 10,000, 280 to 4,000, 200 to 20,900, 280 to 10,000, 200 to 7000, 200 to 2000, 280 to 2900, 280 to 1700, and 200 to 500.

In an embodiment of the invention, any of the polyolefins produced have a molecular weight dispersity (M_{w}/Mₙ, or MWD) of greater than 1 and less than 5, preferably less than 4, more preferably less than 3, more preferably less than 2.5, and most preferably less than 2.

In an embodiment of the invention, any polyolefin produced may have a pour point, as measured by ASTM D 97, of less than 10° C., preferably less than 0° C., preferably less than −10° C., preferably less than −20° C., preferably less than −25° C., preferably less than −30° C., preferably less than −35° C., preferably less than −50° C., and most preferably less than −70° C.

In an embodiment of the invention, any polyolefin produced may have a kinematic viscosity at 40° C. from about 4 to about 80,000 cSt, as measured by ASTM D 445, preferably from about 5 cSt to about 50,000 cSt. In an embodiment of the invention, any polyolefin produced may have a kinematic viscosity at 100° C., as measured by ASTM D 445, from about 1.5 cSt to about 5,000 cSt, from about 2 cSt to about 3,000 cSt, from about 3 cSt to about 1,000 cSt, from about 2 cSt to about 500 cSt, from about 8 cSt to 500 cSt, and from 3.2 cSt to 300 cSt.

The M_{w} and Mₙ were measured by GPC using polystyrene as the calibration standard. The Mₙ was correlated with the fluid viscosity according to a power equation, Mₙ=A×(V)^{B}, wherein V is kinematic viscosity measured at 100° C. according to ASTM D 445 and A and B are constants which vary slightly depending on the type of olefin feeds. For example, when a set polyolefin made from a mixed feed of 33 wt % C₆ and 67 wt % C₁₂ LAOs was analyzed by GPC, the relationship of Mₙ versus 100° C. viscosity was as follows: Mₙ=344.96×(V)^{0.4921}.

Kinematic viscosity (KV) was measured according to ASTM D445 at the temperature indicated (e.g., 100° C. or −40° C.).

Pour point was determined according to ASTM D5950.

Oligomer distribution was determined by using the Hewlett Packard (HP) 5890 Series II Plus GC, equipped with flame ionization detector (FID) and capillary column.

### Applications

The lubricating oils or greases of the present invention are particularly preferred as lubricants of rolling element bearings (e.g., ball bearings), gears, circulation lubrication systems, hydraulics, gas or liquid compressors (such as reciprocating, rotary and turbo-type air compressors, gas turbine compressors, or refrigerator compressors), vacuum pump or metal working machinery, as well as electrical applications, such as for lubrication of electrical switch that produces an electrical arc during on-off cycling or for electrical connectors.

The lubricant or grease components disclosed in this invention are most suitable for applications in industrial machinery where one of more the following characteristics are desirable: wide temperature range, stable and reliable operation, superior protection, extended operation period, energy efficient. These oils are characterized by an excellent balance of performance properties including superior high and low temperature viscosities, flowability, excellent foam properties, shear stability, improved anti-wear characteristics, thermal and oxidative stability, low friction, and low traction. They may find utility as gear oils, bearing oils, circulating oils, compressor oils, hydraulic oils, turbine oils, greases for all kinds of machinery, as well as in other applications, for example, in wet clutch systems, blower bearings, wind turbine gear box, coal pulverizer drives, cooling tower gearboxes, kiln drives, paper machine drives and rotary screw compressors.

Unless stated otherwise, the meanings of terms herein shall be their ordinary meaning in the art. Reference shall be taken, in particular, to Synthetic Lubricants and High-Performance Functional Fluids, Second Edition, Edited by Leslie R. Rudnick and Ronald L. Shubkin, Marcel Dekker (1999). This reference, as well as all patents and applications, test procedures (such as ASTM methods and the like), and other documents cited herein are fully incorporated by reference to the extent such disclosures are not inconsistent with this invention and for all jurisdictions in which such incorporation is permitted. Note that trade names used herein are indicated by a ™ symbol or ® symbol, indicating that the names may be protected by certain trademark rights, e.g., they may be registered trademarks in various jurisdictions. Note also that when numerical lower limits and numerical upper limits are listed herein, ranges from any lower limit to any upper limit are contemplated.

What is claimed is:

1. A method for preparing a liquid polyolefin, the method comprising:
a) contacting a feedstock comprising at least one olefin monomer with a catalyst system comprising at least one activated metallocene catalyst in a polymerization reactor to produce a reactor effluent stream comprising at least one liquid polyolefin, residual catalyst, and unreacted olefin monomer;
b) contacting the reactor effluent stream with a sorbent;
c) filtering the reactor effluent stream through a filter wherein at least a portion of the residual catalyst is removed from the effluent stream, wherein the filtering creates a filter cake comprising at least one sorbent, residual catalyst, and at least one liquid polyolefin;
d) washing the filter cake with a wash fluid comprising at least one hydrocarbon liquid, wherein the wash fluid is at a temperature of about 25° C. to about 60° C.;
e) during the wash step, separating at least a portion of the wash fluid and sending said portion of the wash fluid through a separation process; and
f) recovering at least a portion of the at least one liquid polyolefin from the filter cake and wash fluid.

2. The process of claim 1, wherein the hydrocarbon liquid in the wash fluid is at least one C₅ to C₃₀ hydrocarbon liquid.

3. The process of claim 1, wherein the hydrocarbon liquid in the wash fluid comprises 90 to 100 vol % of at least one hydrocarbon liquid selected from the group comprising C₁₀, C₁₂, and C₁₄.

4. The process of claim 1, wherein the hydrocarbon liquid in the wash fluid corresponds to any of the at least one olefin monomers in the feedstock.

5. The process of claim 1, wherein the wash fluid comprises water, steam, or nitrogen.

6. The process of claim 1, wherein the wash fluid contains no more than 5.0 vol % of water.

7. The process of claim 1, wherein the wash fluid composition is maintained at a steady state concentration during the wash step.

8. The process of claim 7, wherein the steady state concentration of the wash fluid is at least 50 wt % of at least one hydrocarbon liquid selected from the group comprising C₁₀, C₁₂, and C₁₄.

9. The process of claim 7, wherein the steady state concentration of the wash fluid is less than 10 wt % of C_{z}—C₆ hydrocarbons.

10. The process of claim 1, wherein the at least one olefin monomer comprises at least one C₅ to C₃₀ olefin.

11. The process of claim 1, wherein the feedstock is a mixed feedstock comprising C₅ to C₁₄ alpha-olefins.

12. The process of claim 1, wherein the catalyst system comprises a single site metallocene catalyst, an NCA activator, and a trialkyl aluminum co-activator.

13. The process of claim 1, wherein the filter comprises a pressure leaf filter.

14. The process of claim 1, wherein the washed filter cake contains less than 20 wt % of residual polyolefin product upon disposal.

15. The process of claim 1, wherein unreacted olefin monomers, hydrocarbon liquid, and liquid polyolefin are separated from the wash fluid in the separation process.

16. The process of claim 1, wherein at least a portion of the unreacted olefin monomers is recycled to the reactor and/or to the filter.

17. The process of claim 1, wherein at least a portion of the hydrocarbon liquid is recycled to the reactor and/or to the filter.

18. The process of claim 1, the process further including the steps of:
(a) prior to filtering, contacting the reactor effluent stream with a deactivator to deactivate the residual catalyst to produce a deactivated reactor effluent stream; and
(b) contacting the deactivated reactor effluent stream with a sorbent, wherein the sorbent is capable of chemically and physically interacting with the residual catalyst.

19. A lubricant comprising a liquid polyolefin made by the process of claim 1, wherein the lubricant is a gear oil, bearing oil, circulating oil, compressor oil, hydraulic oil, turbine oil, or machinery grease.

20. The lubricant of claim 19, further comprising using the lubricant in a wet gearbox, clutch system, blower bearing, wind turbine gear box, coal pulverizer drive, cooling tower gear box, kiln drive, paper machine drive, or rotary screw compressor.
